(19) United States
(12) Patent Application Publication
(10) Pub. No.: US 2003/0028919 A1
(43) Pub. Date: Feb. 6, 2003

(54) Transgenic trees having increased resistance to imidazolinone herbicides

(76) Inventors: David F. Karnosky, Chassell, MI (US); Gopi K. Podila, Houghton, MI (US); Bixia Xiang, Houghton, MI (US)

Correspondence Address: SUTHERLAND ASBILL & BRENNAN LLP, 999 Peachtree Street, NE, Atlanta, GA, 30309-3996, US

(21) Appl. No.: 10/057,609

(22) Filed: Jan. 24, 2002

Related U.S. Application Data
(60) Provisional application No. 60/264,216, filed on Jan. 25, 2001.

(51) Int. Cl. A01H 7/00; C12N 5/04; A01N 25/00

(52) U.S. Cl. 800/278; 435/419; 504/116.1; 800/319

(57) ABSTRACT

The present invention is directed to transgenic trees having increased resistance to an imidazolinone herbicide. In particular, the present invention relates to larch and poplar trees transformed with an altered AHAS gene, wherein such transformation results in the trees' increased resistance to an imidazolinone herbicide.

### CROSS-REFERENCE TO RELATED APPLICATIONS

This application claims the priority benefit of U.S. Provisional Application Serial No. 60/264,216 filed Jan. 25, 2001.

### FIELD OF THE INVENTION

The present invention relates in general to trees having an increased resistance to imidazolinone herbicides. More specifically, the present invention relates to transgenic trees comprising an altered AHAS gene that have an increased resistance to imidazolinone herbicides.

### BACKGROUND OF THE INVENTION

Imidazolinone and sulfonylurea herbicides are widely used in modem agriculture due to their effectiveness at very low application rates and relative non-toxicity in animals. Imidazolinone and sulfonylurea herbicides inhibit the activity of acetohydroxyacid synthase (AHAS), or acetolactate synthase (ALS) (E.C.4.1.3.18), the key enzyme in the biosynthesis of branch chain amino acids such as valine, leucine and isoleucine (Shaner et al., 1984 Plant Physiol. 76:545-546). By inhibiting AHAS activity, this class of herbicides prevents further growth and development of susceptible plants including many weed species. Several examples of imidazolinone herbicides that are commercially available are PURSUIT® (imazethapyr), CADRE® (imazameth), RAPTOR® (imazamox), ASSERT® (imazethabenz), SCEPTER® (imazaquin) and ARSENAL® (imazapyr). Examples of sulfonylurea herbicides are chlorsulfuron, metsulfuron methyl, sulfometuron methyl, chlorimuron ethyl, thifensulfuron methyl, tribenuron methyl, bensulfuron methyl, nicosulfuron, ethametsulfuron methyl, rimsulfuron, triflusulfuron methyl, triasulfuron, primisulfuron methyl, cinosulfuron, amidosulfuron, fluzasulfuron, imazosulfuron, pyrazosulfuron ethyl and halosulfuron.

Due to their high effectiveness and low-toxicity, imidazolinone herbicides are favored for application by spraying over the top of a wide area of vegetation. The ability to spray an herbicide over the top of a wide range of vegetation decreases the costs associated with plantation establishment and maintenance and decreases the need for site preparation of such chemicals. Spraying over the top of desired tolerant species also results in the ability to achieve maximum yield potential of the desired species due to the absence of competitive species. However, the ability to use such spray over techniques is dependent upon the presence of imidazolinone herbicide resistant species of the desired vegetation in the spray over area.

Among the major agricultural crops, leguminous species such soybean are naturally resistant to imidazolinone herbicides due to their ability to rapidly metabolize the herbicide compounds (Shaner and Robinson, 1985 Weed Sci. 33:469-471). Other crops such as corn (Newhouse et al., 1991 Theor. Appl. Genet. 83:65-70), wheat (Newhouse et al., 1992 Plant Physiol. 100:882-886) and rice (Barrette et al., 1989 Crop Safeners for Herbicides, Academic Press New York, pp. 195-220) are somewhat susceptible to imidazolinone herbicides. The differential sensitivity to the imidazolinone herbicides is dependent on the chemical nature of the particular herbicide and differential metabolism of the compound from a toxic to a non-toxic form in each plant (Shaner et al., 1984 Plant Physiol. 76:545-546; Brown et al., 1987 Pestic. Biochm. Physiol. 27:24-29). Other plant physiological differences such as absorption and translocation also play an important role in sensitivity (Shaner and Robinson, 1985 Weed Sci. 33:469-471).

Computer-based modeling of the three dimensional conformation of the AHAS-inhibitor complex predicts several amino acids in the proposed inhibitor binding pocket as sites where introduced mutations would likely confer selective resistance to imidazolinones (Ott et al., 1996 J. Mol. Biol. 263:359-368). Transgenic plants produced with these rationally designed mutations in the proposed binding sites of the AHAS enzyme have in fact exhibited specific resistance to a single class of herbicides (Ott et al., 1996 J. Mol. Biol. 263:359-368). Other mutations in the AHAS gene have been linked to resistance to the imidazolinone herbicides in canola (Swanson et al., 1989 Theor. Appl. Genet. 78:525-530) and corn (Newhouse et al., 1991 Theor. Appl. Genet. 83:65-70).

Studies of the ALS gene in other crop plants have also resulted in sulfonylurea and imidazolinone resistance in those plants. In one report, use of a mutant ALS gene from Arabidopsis coupled with selection on sulfonylurea herbicide resulted in the production of resistant transgenic rice plants (Li et al., 1992 Plant Cell Rep. 12:250-255). An increase in in vitro resistance to chlorsulfuron of similar magnitude (200-fold) was demonstrated in transgenic rice containing a 35S/ALS transgene (Li et al., 1992 Plant Cell Rep. 12:250-255), and imidazolinone-resistant growth of transgenic tobacco was reported to be 100-fold greater than non-transformed control plants (Sathasivan et al., 1991 Plant Physiol. 97:1044-1050).

Expression of the introduced AHAS or ALS gene at different magnitudes has also been achieved by manipulating several aspects of the transformation including the use of different promoters and screening larger populations of transformants (Odell et al., 1990 Plant Physiol. 94:1647-1654; Sathasivan et al., 1991 Plant Physiol. 97:1044-1050; Li et al., 1992 Plant Cell Rep. 12:250-255). Studies showed that replacing the Arabidopsis ALS promoter with the CaMV35S promoter resulted in 40-fold differences in in vitro resistance to chlorsulfuron (Li et al., 1992 Plant Cell Rep. 12:250-255). In tobacco, increases in resistance to imazethapyr in individual calli transformed with a mutant ALS gene from Arabidopsis ranged from 10- to 1000-fold, most likely reflecting the differences in gene copy numbers or in chromosomal positions of the transgenes (Sathasivan et al., 1991 Plant Physiol. 97:1044-1050).

Plant resistance to imidazolinone has also been reported in a number of patents. U.S. Pat. No. 4,761,373 generally describes the use of an altered AHAS gene to elicit herbicide resistance in plants, and specifically discloses certain imidazolinone resistant corn lines. U.S. Pat. No. 5,013,659 discloses mutants exhibiting herbicide resistance possessing mutations in at least one amino acid in one or more conserved regions. The mutations described therein encode either cross-resistance for imidazolinones and sulfonylureas or sulfonylurea-specific resistance but imidazolinone-specific resistance is not described. Additionally, U.S. Pat. No. 5,731,180 and U.S. Pat. No. 5,767,361 discuss an isolated gene having a single amino acid substitution in a wild-type monocot AHAS amino acid sequence that results in imidazolinone-specific resistance.

However, to date, the prior art has not described the successful transformation of a tree with an altered AHAS gene resulting in an increased resistance to imidazolinone herbicides. In fact, trees in general are quite recalcitrant to transformation. Poplar species may be excepted from this general group as they are more receptive to transformation. But even poplars vary considerably from species to species and genotype to genotype such that specific protocols of transformation must be adopted for each. References discussing transformation of trees include the following: Tsai et al., 1994 Plant Cell Reports 14:94-97; Huang et al., 1991 In Vitro Cell. Dev. Biol. 270:201-207; and Shin et al., 1994 Can. J. For. Res. 24:2059-2067.

Therefore, what are needed in the art are transgenic trees that are transformed with an altered AHAS gene and that thereby have increased resistance to imidazolinone herbicides. Also needed in the art are methods for producing transgenic trees having increased resistance to imidazolinone herbicides and seeds produced by those trees.

### SUMMARY OF THE INVENTION

The present invention relates to transgenic trees having increased resistance to an imidazolinone herbicide. In particular, the present invention relates to trees transformed with an altered AHAS gene, wherein such transformation results in the tree's increased resistance to an imidazolinone herbicide. The imidazolinone herbicide can be selected from, but is not limited to, the group consisting of imazethapyr, imazapic, imazamox, imazaquin, imazethabenz and imazapyr.

One embodiment of the invention provides a transgenic tree cell transformed by an altered AHAS nucleic acid, wherein expression of the nucleic acid in the tree cell results in increased resistance to an imidazolinone herbicide as compared to a wild type variety of the tree cell. The altered AHAS nucleic acids described herein include a nucleic acid having a sequence as shown in SEQ ID NO:2 that is derived from imidazolinone resistant Arabidopsis thaliana. Also provided herein are transgenic tree parts, transgenic tree seeds and transgenic trees comprising a tree cell transformed with an altered AHAS nucleic acid and thereby having increased resistance to an imidazolinone herbicide as compared to a wild type variety of the tree part, tree seed and tree, respectively. Preferably, the tree part, tree seed and tree are true breeding for an increased resistance to an imidazolinone herbicide.

The transgenic trees having increased resistance to an imidazolinone herbicide as described herein can be of any genus or species, however, the tree is preferably a member of a genus selected from the group consisting of Larix genus and Populus genus. More preferably, the tree is either a Larix species selected from the group consisting of Larix decidua, Larix lyallii, Larix kaempferi, Larix laricina, Larix occidentalis and hybrids thereof or a Populus species selected from the group consisting of Populus angustifolia, Populus balsamifera, Populus canadescens, Populus deltoides, Populus fremontii, Populus grandidentata, Populus tremuloides, Populus trichocarpa and hybrids thereof.

The present invention also includes a method of producing a transgenic tree with an increased resistance to an imidazolinone herbicide as compared to a wild type variety of the tree comprising, transforming a tree cell with an expression cassette comprising an altered AHAS nucleic acid, and generating from the tree cell a tree with an increased resistance to an imidazolinone herbicide. Preferably, the expression cassette further comprises a transcription initiation regulatory region that is functional in the tree cell and a translation initiation regulatory region that is functional in the tree cell.

### BRIEF DESCRIPTION OF THE DRAWINGS

FIGS. 1A-1C show the genomic polynucleotide sequence (SEQ ID NO:1), the coding polynucleotide sequence (SEQ ID NO:2) and amino acid (SEQ ID NO:3) sequence of an altered AHAS gene.

FIG. 2 is a schematic representation of the pAC1550-AHAS plasmid.

FIGS. 3A-3C are schematic representations of relevant portions of vectors described herein. FIG. 3A is a schematic representation of the intermediate vector pOEA1. FIG. 3B is a schematic representation of the binary vector pTCS5-AHAS. FIG. 3C is a schematic representation of the altered AHAS gene construct.

### DETAILED DESCRIPTION

The present invention is directed to transgenic trees, tree parts and tree cells comprising an altered AHAS gene and having increased resistance to imidazolinone herbicides. The present invention also includes seeds produced by the transgenic trees described herein, wherein the trees are true breeding for an increased resistance to an imidazolinone herbicide as compared to a wild type variety of the tree. Also described herein is a method of producing a transgenic tree with an increased resistance to an imidazolinone herbicide as compared to a wild type variety of the tree comprising, transforming a tree cell with an expression cassette comprising an altered AHAS nucleic acid, and generating from the tree cell a tree with an increased resistance to an imidazolinone herbicide.

As used herein, the term “tree” refers to a woody plant having a single, erect and persistent stem of at least 10 centimeters in diameter, measured at 1.3 meters above the mean ground level, and with a total height of at least 5 meters. A crown of leaves may be more or less well defined. The transgenic trees of the present invention can include, but are not limited to, aspen, douglas-fir, conifer, eucalyptus, larch, loblolly pine, poplar, radiata pine and spruce.

In one embodiment, the tree is a member of a genus selected from the group consisting of the Larix genus and the Populus genus. In a preferred embodiment, the tree is a Larix species selected from the group consisting of Larix decidua (European larch), Larix lyallii (alpine larch), Larix kaempferi (Japanese larch), Larix laricina (tamarack), Larix occidentalis (western larch) and hybrids thereof. Preferred hybrids include 2- and 3-way hybrids of Larix decidua, Larix kaempferi and/or Larix laricina. In a further preferred embodiment, the tree is a hybrid of Larix decidua and Larix kaempferi. In another preferred embodiment, the tree is a Populus species selected from the group consisting of Populus angustifolia (narrow leaf cottonwood), Populus balsamifera (balsam poplar), Populus canadescens, Populus deltoides (eastern cottonwood), Populus fremontii (freemont cottonwood), Populus grandidentata (big tooth aspen), Populus tremuloides (quaking aspen), Populus trichocarpa (black cottonwood) and hybrids thereof. Preferred hybrids include 2- and 3-way hybrids of Populus canadescens, Populus tremuloides, Populus deltoides and/or Populus grandidentata. In a further preferred embodiment, the tree is a hybrid of Populus canadescens and Populus grandidentata.

The imidazolinone herbicide can be selected from, but is not limited to, PURSUIT® (imazethapyr), CADRE® (imazapic), RAPTOR(® (imazamox), SCEPTER® (imazaquin), ASSERT® (imazethabenz), ARSENAL® (imazapyr), a derivative of any of the aforementioned herbicides, or a mixture of two or more of the aforementioned herbicides, for example, imazapyr/imazamox (ODYSSEY®). More specifically, the imidazolinone herbicide can be selected from, but is not limited to, 2-(4-isopropyl-4-methyl-5-oxo-2-imidiazolin-2-yl)-nicotinic acid, 2-(4-isopropyl)-4-methyl-5-oxo-2-imidazolin-2-yl)-3-quinolinecarboxylic acid, 5-ethyl-2-(4-isopropyl-4-methyl-5-oxo-2-imidazolin-2-yl)-nicotinic acid, 2-(4-isopropyl-4-methyl-5-oxo-2-imidazolin-2-yl)-5-(methoxymethyl)-nicotinic acid, 2-(4-isopropyl-4-methyl-5-oxo-2-imidazolin-2-yl)-5-methylnicotinic acid, and a mixture of methyl 6-(4-isopropyl-4-methyl-5-oxo-2-imidazolin-2-yl)-m-toluate and methyl 2-(4-isopropyl-4-methyl-5-oxo-2-imidazolin-2-yl)-p-toluate. As used herein, the term “derivative” encompasses any herbicide that inhibits the activity of an acetohydroxyacid synthase (AHAS) protein.

The methods of the present invention involve the introduction of an altered AHAS nucleic acid into a tree cell, either maintained on a separate plasmid or integrated into the genome of the tree cell. If integrated into the genome, such integration can be random, or it can take place by recombination such that the native nucleic acid sequence is replaced by the introduced copy, or by using an altered AHAS nucleic acid in trans such that the nucleic acid is functionally linked to a functional expression unit containing at least a sequence facilitating the expression of a nucleic acid and a sequence facilitating the polyadenylation of a functionally transcribed nucleic acid.

Preferably, an expression cassette comprising a transcription initiation regulatory region that is functional in a tree cell, a translation initiation regulatory region that is functional in a tree cell, and an altered AHAS nucleic acid, is integrated into the genome of the tree cell. The present invention also contemplates the introduction of other nucleic acids into a tree host cell along with an altered AHAS nucleic acid. In particular, the present invention includes a transgenic tree comprising an altered AHAS nucleic acid and one or more nucleic acids encoding structural proteins that confer disease resistance, insect resistance, sterility, improved pulp or improved wood quality.

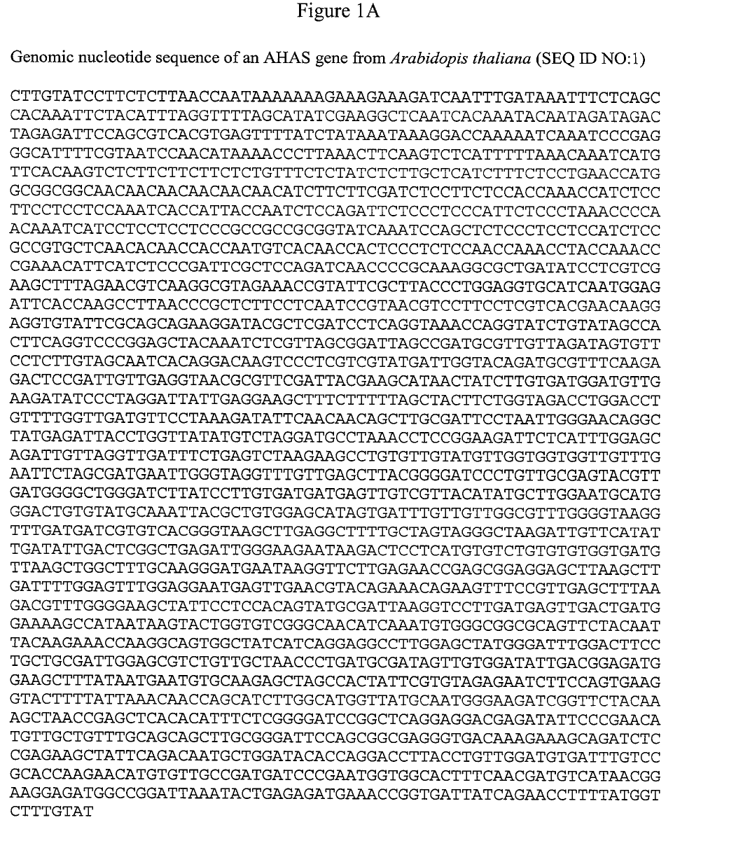

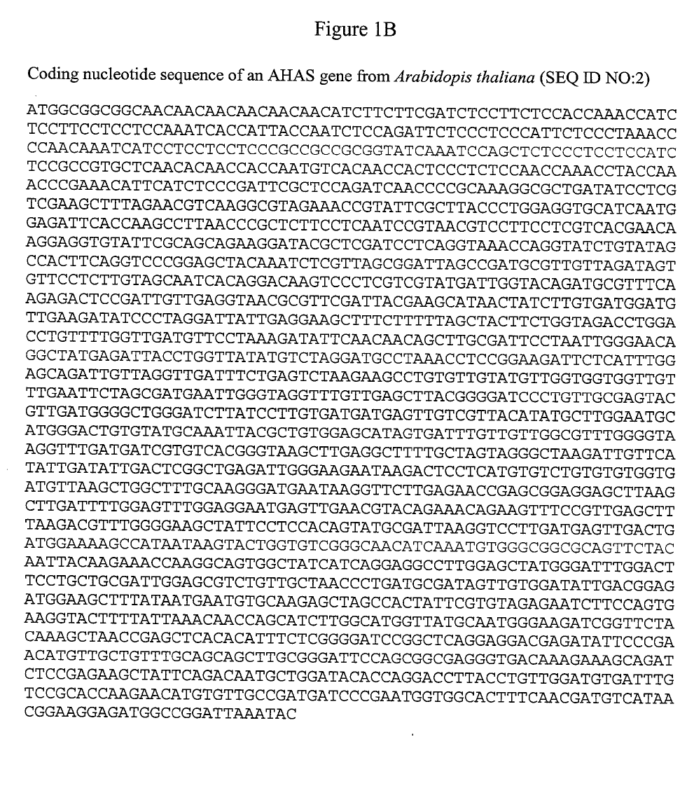

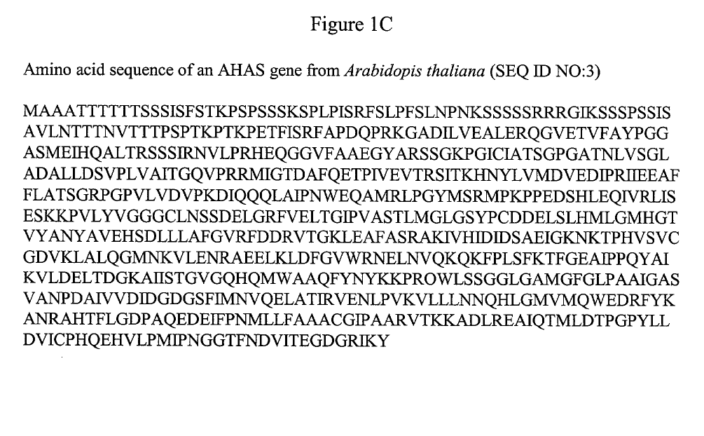

As used herein, the term “altered AHAS nucleic acid” refers to any AHAS nucleic acid that is mutated from a wild type AHAS nucleic acid and that confers increased imidazolinone resistance to a tree into which it is transformed. In a preferred embodiment, the altered AHAS nucleic acid comprises the polynucleotide sequence shown in SEQ ID NO:2 and FIG. 1B. SEQ ID NO:2 represents the coding region of the entire nucleotide sequence shown in SEQ ID NO:1 and FIG. 1A. SEQ ID NO:1 represents the entire altered AHAS nucleic acid including non-coding regions. The sequence shown in SEQ ID NO:1 is derived from an imidazolinone resistant mutant of Arabidopsis thaliana described in U.S. Pat. No. 5,767,366 to Sathasivan et al.

By “AHAS nucleic acid” is meant a RNA or DNA sequence that encodes or directs the expression of an AHAS protein, and may include a coding region and its corresponding untranslated 5′ and 3′ sequence regions. Additionally, “AHAS gene” refers specifically to a DNA sequence that encodes or directs the expression of an AHAS protein. By “AHAS protein” is meant an acetohydroxyacid synthase protein and by “altered AHAS protein” is meant any AHAS protein that is mutated from a wild type AHAS protein and that confers increased imidazolinone resistance to a tree, tree cell, tree part or tree tissue when it is expressed therein. In a preferred embodiment, the altered AHAS protein comprises the amino acid sequence shown in SEQ ID NO:3 and FIG. 1C. SEQ ID NO:3 is the amino acid sequence corresponding to the nucleotide coding region shown in SEQ ID NO:2.

While it is preferable that the altered AHAS nucleic acid encodes an AHAS protein comprising an amino acid sequence as shown in SEQ ID NO:3, the present invention also includes a tree, tree cell or tree part comprising a polynucleotide comprising SEQ ID NO:1; a polynucleotide comprising SEQ ID NO:2; a polynucleotide encoding a polypeptide comprising SEQ ID NO:3; a polynucleotide comprising at least 60 consecutive nucleotides of any of the aforementioned polynucleotides or a polynucleotide complementary to any of the aforementioned polynucleotides.

It is to be understood that the present invention encompasses a transgenic tree comprising any altered AHAS nucleic acid. Accordingly, the present invention also includes transgenic trees comprising a homolog of the amino acid sequence shown in SEQ ID NO:3 or the nucleotide sequence shown in SEQ ID NO:1 or SEQ ID NO:2, wherein expression of the homolog results in an increased resistance to an imidazolinone herbicide in the tree. Homologs can differ from the amino acid sequence shown in SEQ ID NO:3 by amino acid sequence differences, post-translational modifications or by both. Preferably, an altered AHAS protein encoded by an altered AHAS nucleic acid sequence described herein is at least about 50-60% homologous, more preferably 60-70%, even more preferably 70-80%, 80-90%, 90-95%, and most preferably, 96%, 97%, 98% or 99% homologous to a protein having the sequence shown in SEQ ID NO:3. In yet another embodiment, the altered AHAS amino acid homologs included in the present invention are at least about 50-60%, preferably at least about 60-70%, and more preferably at least about 70-75%, 75-80%, 80-85%, 85-90% or 90-95%, and most preferably at least about 96%, 97%, 98%, 99% or more identical to an entire amino acid sequence encoded by a nucleic acid sequence shown in SEQ ID NO:2. The length of sequence comparison is at least 15 amino acid residues, preferably at least 25 amino acid residues, and more preferably at least 35 amino acid residues. Post-translational modifications include in vivo and in vitro chemical derivatization of polypeptides, e.g., acetylation, carboxylation, phosphorylation, or glycosylation, and such modifications may occur during polypeptide synthesis or processing or following treatment with isolated modifying enzymes. Additionally, the differences in amino acid sequences between the amino acid sequence shown in SEQ ID NO:3 and its homolog can be natural (i.e., found in nature) or induced.

Also included in the present invention are transgenic trees comprising homologs of the altered AHAS nucleic acids described herein. Homologs of the altered AHAS nucleic acids comprise a nucleotide sequence which is at least about 50-60%, preferably at least about 60-70%, more preferably at least about 70-75%, 75-80%, 80-85%, 85-90% or 90-95%, and even more preferably at least about 95%, 96%, 97%, 98%, 99% or more identical to a nucleotide sequence shown in SEQ ID NO:2, or to a portion comprising at least 60 consecutive nucleotides thereof. The preferable length of sequence comparison for nucleic acids is at least 75 nucleotides, more preferably at least 100 nucleotides and most preferably the entire length of the coding region. It is preferred that the homologs confer increased resistance to imidazolinone herbicides in a tree when expressed therein.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleic acids at corresponding amino acid positions or nucleic acid positions are then compared. When a position in the first sequence is occupied by the same amino acid residue or nucleic acid as the corresponding position in the second sequence, then the molecules are identical at that position. The percent identity between the two sequences is a function of the number of identical positions shared by the sequences. For example, the percent identity equals the number of identical positions divided by the number of total positions times one hundred. The two compared sequences are preferably the same length.

For the purposes of the invention, the percent sequence identity between two nucleic acid or polypeptide sequences is determined using the Vector NTI 6.0 (PC) software package (InforMax, 7600 Wisconsin Ave., Bethesda, Md. 20814). A gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. It is to be understood that for the purposes of determining sequence identity when comparing a DNA sequence to an RNA sequence, a thymidine nucleotide is equivalent to a uracil nucleotide.

Nucleic acid molecules encoding homologs and fragments of the amino acid sequence shown in SEQ ID NO:3 can be isolated based on the nucleic acid's ability to hybridize under stringent conditions to SEQ ID NO:1 or SEQ ID NO:2. Therefore, the present invention includes a transgenic tree comprising a nucleic acid that hybridizes to the sequence shown in SEQ ID NO:1 or SEQ ID NO:2, wherein expression of the nucleic acid in the tree results in the tree's increased resistance to an imidazolinone herbicide. More preferably, the nucleic acid is at least 15 nucleotides in length and hybridizes to the sequence shown in SEQ ID NO:1 or SEQ ID NO:2 under stringent conditions or highly stringent conditions.

As used herein with regard to hybridization for DNA to DNA blot, the term “stringent conditions” refers to hybridization overnight at 60° C. in 10× Denhart's solution, 6×SSC, 0.5% SDS and 100 μg/ml denatured salmon sperm DNA. Blots are washed sequentially at 62° C. for 30 minutes each time in 3×SSC/0.1% SDS, followed by 1×SSC/0.1% SDS and finally 0.1×SSC/0.1% SDS. As also used herein, “highly stringent conditions” refers to hybridization overnight at 65° C. in 10× Denhart's solution, 6×SSC, 0.5% SDS and 100 μg/m 1 denatured salmon sperm DNA. Blots are washed sequentially at 65° C. for 30 minutes each time in 3×SSC/0.1% SDS, followed by 1×SSC/0.1% SDS and finally 0.1×SSC/0.1% SDS. Methods for nucleic acid hybridizations are described in Meinkoth and Wahl, 1984 Anal. Biochem. 138:267-284; Current Protocols in Molecular Biology, Chapter 2, Ausubel et al. Eds., Greene Publishing and Wiley-Interscience, New York, 1995; and Tijssen, Laboratory Techniques in Biochemistry and Molecular Biology: Hybridization with Nucleic Acid Probes, Part I, Chapter 2, Elsevier, New York, 1993. Preferably, a nucleic acid molecule of the invention that hybridizes under stringent or highly stringent conditions to a sequence of SEQ ID NO:2 corresponds to a naturally occurring nucleic acid molecule. As used herein, a “naturally occurring” nucleic acid molecule refers to an RNA or DNA molecule having a nucleotide sequence that occurs in nature (e.g., encodes a natural polypeptide).

An isolated nucleic acid molecule encoding an altered AHAS protein having a sequence that differs from that of SEQ ID NO:3 can be created by introducing one or more nucleic acid substitutions, additions or deletions into the nucleic acids encoding those altered AHAS proteins. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g. glycine, asparagines, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted, nonessential amino acid residue in an AHAS protein is preferably replaced with another amino acid residue from the same side chain family.

The present invention encompasses not only transgenic tree cells comprising full-length altered AHAS proteins, but also includes transgenic tree cells comprising altered AHAS protein fragments. As used herein, the term “fragment” means at least 20 contiguous amino acids, preferably at least 30 contiguous amino acids, more preferably at least 50 contiguous amino acids, and most preferably at least 60 to 80 or more contiguous amino acids. Fragments of altered AHAS proteins can be generated by methods known to those of ordinary skill in the art or may result from normal protein processing (e.g. removal of amino acids from the nascent polypeptide that are not required for biological activity or removal of amino acids by alternative mRNA splicing or alternative protein processing events). In a preferred embodiment, an altered AHAS protein fragment maintains the ability to confer increased imidazolinone resistance to a tree, tree cell, tree part or tree tissue in which it is produced.

In producing a transgenic tree of the present invention, an expression cassette containing the introduced nucleic acid can be inserted into protoplasts, intact tissues, such as immature embryos and meristems, callus cultures or isolated cells. Preferably, expression cassettes are inserted into intact tissues. Methods for introducing a nucleic acid sequence into a tree are described, for example, in Strauss, S. H. et al. 1995 Genetic Engineering of Reproductive Sterility in Forest Trees. Mole. Breed. 1:5-26. Methods for introducing an expression cassette into tree tissue include the direct infection or cocultivation of tree tissue with Agrobacterium tumefaciens (Horsch et al., 1985 Science 227:1229) or Agrobacterium rhizogenes (Shin et al., 1994 Can. J. For. Res. 24:2059-2067).

Preferably, a disarmed Ti-plasmid is used as a vector for inserting an altered AHAS gene into a tree cell. The present invention specifically describes a Ti-plasmid containing an altered AHAS gene expression cassette and the integration of the altered AHAS gene expression cassette into a tree host cell genome. As used herein, the term “expression cassette” refers to a DNA sequence comprising a transcriptional initiation regulatory region and a translational initiation regulatory region, both capable of being recognized by the host cell. As described herein, an altered AHAS gene expression cassette is an expression cassette further comprising an altered AHAS gene. In addition to an altered AHAS gene, the expression cassettes described herein can contain other structural genes such as selection markers conferring antibiotic resistance or other selectable traits. Preferably, the altered AHAS expression cassette is inserted adjacent to the T-DNA right border, or between the T-DNA left and right borders, of the Ti-plasmid. Following genetically engineered insertion of an altered AHAS gene expression cassette into T-DNA in Agrobacterium, the host tree, or tree cell, can be transformed by the bacterium or Ti plasmid, thus inserting the altered AHAS nucleic acid into the host tree cell chromosome to eventually produce a transgenic tree comprising an altered AHAS nucleic acid.

To provide for transcription of the altered AHAS gene in the tree host cell, a variety of transcriptional initiation regions can be employed. The transcriptional initiation regions can be constitutive, inducible, developmental stage-preferred, cell type-preferred, tissue-preferred or organ-preferred. In particular, the transcriptional and translational regulatory regions from Agrobacterium tumefaciens can be employed. Examples of other transcriptional initiation regions that can be used, include, but are not limited to, opine promoters, such as the octopine synthase promoter, nopaline synthase promoter, agropine synthase promoter and mannopine synthase promoter, viral promoters such as CaMV region VI promoter and full length (35S) promoter and promoters associated with the ribulose-1, 5 bisphosphate carboxylase genes.

In one embodiment, the transcriptional initiation region is joined to the altered AHAS gene to provide for transcriptional initiation upstream from the initiation codon, normally within about 200 bases of the initiation codon, where the untranslated 5′-region lacks an ATG. The 3′-end of the structural gene will have one or more stop codons that are joined to a transcriptional termination region functional in the tree host cell. The termination region can be associated with the altered AHAS gene or another structural gene such as an antibiotic resistance gene.

Accordingly, also described herein are recombinant expression vectors and recombinant expression cassettes comprising the altered AHAS nucleic acid sequences described herein and host cells into which such expression cassettes have been introduced. As used herein, the term “recombinant AHAS protein” refers to an altered AHAS protein expressed by a recombinant expression vector or recombinant expression cassette comprising an altered AHAS nucleic acid sequence. The host cell can be derived from bacteria, yeast or tree material. Preferably the host cell is derived from tree material and more preferably the host cell is derived from a rapidly growing tree tissue such as a shoot, root or young leaf. The host cell as described above may be found or located in a tree tissue, a tree organ or a whole tree. It is to be understood that the term “host cell” refers not only to the particular subject cell, but also to the progeny or potential progeny of such a cell. Because certain modifications may occur in succeeding generations due to either mutation or environmental influences, such progeny may not be identical to the parent cell, but are still included within the scope of the term as used herein.

Expression vectors suitable for use in trees, and therefore suitable for use in the present invention, are well known to those of skill in the art. In a preferred embodiment, the expression vector is an Agrobacterium Ti vector and the expression cassette is derived from this vector. Several other examples of suitable expression vectors can be found in Shaner, D. L. and Singh, B. K. 1997. Acetohydroxyacid Synthase Inhibitors in: Herbicide Activity: Toxicology, Biochemistry and Molecular Biology. R M Roe (ed.) IOS Press pp. 69-110.

The present invention additionally includes methods of expressing the altered AHAS proteins described herein in a tree and thereby increasing the tree's resistance to an imidazolinone herbicide. As described above, an altered AHAS nucleic acid is introduced into a host cell and either maintained on a separate plasmid or integrated into the genome of a host cell. Preferably, an altered AHAS gene expression cassette is introduced into a host and an altered AHAS protein expressed therefrom. In one embodiment of the present invention, an altered AHAS nucleic acid described herein is operably linked to a non-AHAS nucleic acid or a heterologous nucleic acid, both hereinafter referred to as a “chimeric altered AHAS nucleic acid sequence.” Within a recombinant expression vector or recombinant expression cassette, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). In other embodiments, the chimeric altered AHAS protein encoded by the chimeric altered AHAS nucleic acid sequence has an activity that differs from that of the altered AHAS protein alone.

Standard techniques for construction of such chimeric altered AHAS nucleic acid sequences are well known to those of ordinary skill in the art and can be found in references such as Sambrook et al., Molecular Cloning: A Laboratory Manual. 2^{nd} ed., Cold Spring Harbor Laboratory, Cold Spring Harbor Laboratory Press, Cold Spring Harbor, N.Y., 1989 (or latest edition). A variety of strategies are available for ligating fragments of DNA, the use of which depends on the nature of the termini of the DNA fragments. One of ordinary skill in the art recognizes that in order for the heterologous nucleic acid sequence to be expressed, the construction of the expression vector requires promoter elements and signals for efficient polyadenylation of the transcript. Accordingly, the altered AHAS regulatory region that contains the consensus promoter sequence known as the TATA box can be ligated directly to a promoterless heterologous coding sequence. Additionally, the 3′ end of a heterologous nucleic acid sequence can be ligated to a termination sequence comprising a polyadenylation site or the polyadenylation site can be provided by the heterologous nucleic acid sequence itself.

As described above, the present invention teaches compositions and methods for increasing the imidazolinone resistance of a tree or seed as compared to a wild-type variety of that tree or seed. The invention includes a method of producing a transgenic tree with an increased resistance to an imidazolinone herbicide as compared to a wild type variety of the tree comprising, transforming a tree cell with an expression cassette comprising an altered AHAS nucleic acid, and generating from the tree cell a tree with an increased resistance to an imidazolinone herbicide. The present invention also includes methods of controlling weeds in the vicinity of a tree comprising, providing a tree containing an altered AHAS nucleic acid and having increased resistance to an imidazolinone herbicide as compared to a wild type variety of the tree, and applying an imidazolinone herbicide to the weeds and the tree. In preferred embodiments, the altered AHAS nucleic acid comprises a polynucleotide sequence as shown in SEQ ID NO:1 or SEQ ID NO:2.

In other preferred embodiments of these methods, the imidazolinone resistance of a tree or seed is increased such that the tree or seed can withstand an imidazolinone herbicide application of approximately one to three pounds of active ingredient per acre. The term “variety” refers to a group of trees within a species that share constant characters that separate them from the typical form and from other possible varieties within that species. While possessing at least one distinctive trait, a variety is also characterized by some variation between individuals within the variety, based primarily on the Mendelian segregation of traits among the progeny of succeeding generations. A variety is considered “true breeding” for a particular trait if it is genetically homozygous for that trait to the extent that, when the true-breeding variety is self-pollinated, a significant amount of independent segregation of the trait among the progeny is not observed. In the present invention, the trait arises from the transgenic expression of a single DNA sequence introduced into a tree variety.

By providing for trees having increased resistance to imidazolinone, a wide variety of formulations can be employed for protecting trees from weeds, so as to enhance tree growth and reduce competition for nutrients. An imidazolinone herbicide can be used by itself for post emergence control of weeds in areas surrounding the transgenic trees described herein or an imidazolinone herbicide formulation can be used that contains other additives. Such additives include other herbicides, detergents, adjuvants, spreading agents, sticking agents, stabilizing agents, or the like. The imidazolinone herbicide formulation can be a wet or dry preparation and can include, but are not limited to, flowable powders, emulsifiable concentrates and liquid concentrates. The imidazolinone herbicide and herbicide formulations can be applied in accordance with conventional methods, for example, by spraying, irrigation, dusting, or the like.

It should be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims. Additionally, all references cited herein are hereby expressly incorporated herein by reference.

### EXAMPLES

### Example 1

Transformation and Selection of Putative Transgenic Larch

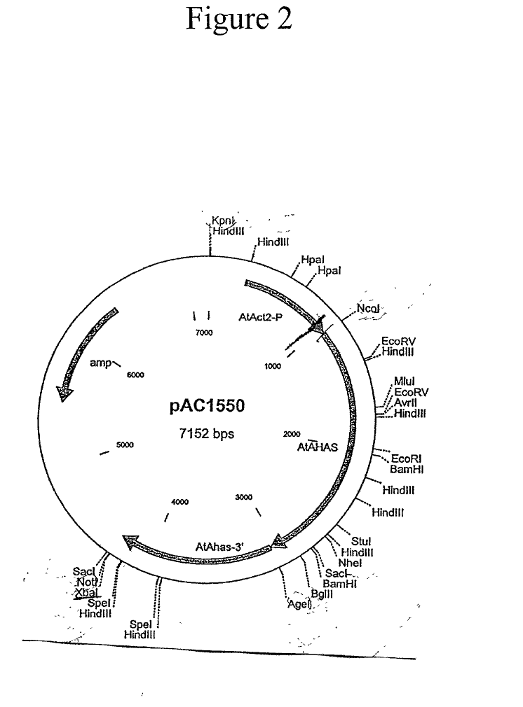

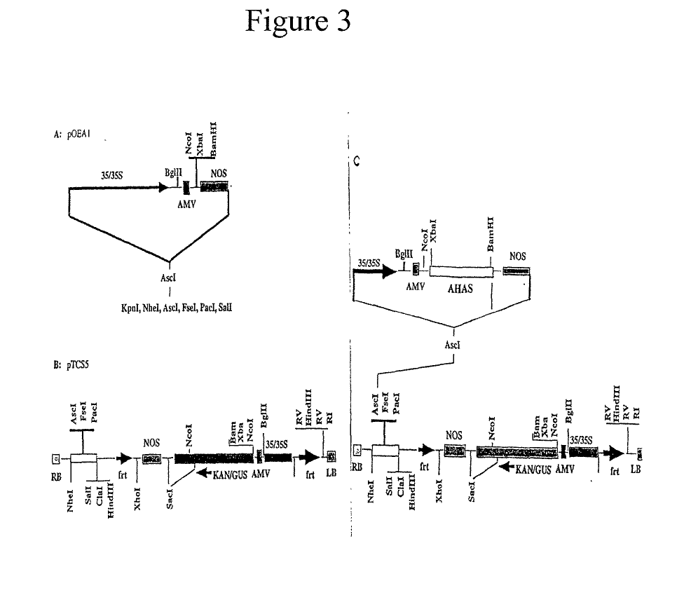

Plasmid pAC1550 as shown in FIG. 2 was obtained containing the altered AHAS gene shown in SEQ ID NO:1. The altered AHAS gene was transferred into a binary pTCS5 vector using an intermediate vector pOEA1. (FIG. 3B). Agrobacterium rhizogenes strain 11325, harboring the oncogenic nopaline-type pRi11325 and the binary vector (pTCS5-AHAS) was maintained at 28° C. on solid AB medium (Chilton et al., 1974 Proc. Natl. Acad. Sci. USA 71:3672-3673) and supplemented with appropriate antibiotics. Two-day old bacterial cultures were used to inoculate approximately 1068 seedlings from a Larix decidua and Larix kaempferi hybrid (hereinafter “larch”).

Prior to inoculation, larch seeds were surface sterilized with 30% aqueous hydrogen peroxide for 10 minutes and rinsed three times with sterile demineralized water. After 6-12 hours, sterilization was repeated. Seeds were germinated in sterile conditions on water solidified with 8 g/L agar. One week after germination, seedlings were removed for inoculation.

After removing the seed coat and gametophyte tissue, a linear wound was made centrally from the shoot apex to the hypocotyls (5-4 mm) with a sterile scalpel blade and the bacterial scrape was placed in the wound. The inoculated seedlings were placed on growth-regulator free, half-strength GD medium (Gresshoff and Doy, 1972 Planta 107:161-170), supplemented with 1% sucrose in Petri dishes and incubated under continuous 3000 lux mixed cool-white fluorescent (70%) and incandescent (30%) illumination at 24° C. for four weeks. Alternatively, four days after inoculation, the inoculated seedlings were transferred to half-strength GD medium supplemented with 1% sucrose and one tablet/L Augmentin 250, containing a mixture of 250 mg amoxillin and 125 mg clavulanic acid (Beecham Laboratories, Bristol, Tenn.) to kill the bacteria. Control seedlings were wounded, but not inoculated with bacteria.

Buds were then excised from parental seedlings and transferred to half-strength GD medium supplemented with one tablet/L Augmentin and 40 mg/L kanamycin sulfate. At times, kanamycin was added to the selection medium after bacteria were completely cleared by two consecutive subcultures on half-strength GD medium containing Augmentin. Subcultures were made at 4-week intervals. Growing shoots were subcultured to LMG medium (Litvay et al., 1981 Wis. Tech. Pap. Ser. 155:1-17) supplemented with 1.46 g/L glutamine and half-strength GD medium, alternately, to stimulate elongation.

Healthy and elongated shoots (2-3 cm) were rooted either spontaneously on the selection medium or on DCR-1 medium containing 0.1 mg/L indol-3-butyric acid (Gupta and Durzan, 1985 Plant Cell Rep. 4:177-179). Rooted plantlets were transplanted to plastic 7.5-cm² pots filled with a soil less mix (peat:perlite:vermiculite, 1:1:1) and kept in a mist chamber for four weeks. They were then moved into greenhouse conditions and transferred to 15 cm diameter plastic pots.

### Example 2

Transformation and Selection of Putative Transgenic Aspen

Plasmid pAC1550 as shown in FIG. 2 was obtained containing the altered AHAS gene shown in SEQ ID NO:1. The altered AHAS gene was transferred into a binary pTCS5 vector using an intermediate vector pOEA1. (FIG. 3B). Agrobacterium tumefaciens was incubated overnight with the plasmid containing the altered AHAS gene (pTCS5-AHAS) in 5 ml LB plus kanamycin (50 mg/L) and gentamycin (20 mg/L). The A. tumefaciens culture was then transferred into 50 ml LB plus kanamycin (50 mg/L), gentamycin (20 mg/L) and AS 20 mM, pH 5.2, for 8-10 hours.

Aspen leaf material (LPI 2, 3, 4) was sterilized with Clorox (10%) for 10 minutes, rinsed with water at least 3 times and blotted to remove excess water. The aspen leaves were then cut into approximately 580 0.5×0.5 cm discs and placed in the bacterial cell suspension for 10-20 minutes. After blotting briefly, the leaf discs were put on agar WPM medium, the plates were inverted and incubated at 28° C. for 48 hours. The leaf discs were washed with water 3-5 times until the water ran clear and then incubated with WPM plus 300 mg/L ticarcillin, 300 mg/L cefotaxime and 20 μM for 2-4 hours with gentle shaking (100 r.p.m.). The leaf discs were again rinsed with water, blotted onto paper and placed onto solid plant medium (WPM with kanamycin 50 mg/L, TDZ 0.5 mg/L and Augmentin 300 mg/L, pH 5.4). The leaf discs were then incubated at 23° C. with a 16-hour photoperiod while changing the medium every 10 days. After two months, shoot regeneration occurred and the shoots were transferred into WPM/kanamycin/Augmentin medium for elongation. Once the shoots grew to 1.5 cm in length, the shoots were transferred into rooting medium containing NAA (0.5 mg/L). Rooted shoots were grown to fill the box and the transgenic plants were transferred into a pot with a mixture of vermiculite:peat:top soil (1:1:1) and placed in the greenhouse.

### Example 3

PCR Analysis of Putative Transgenic Larch and Aspen

PCR analysis was performed using methods known to those of skill in the art. The specific primers used for amplification of the altered AHAS gene sequence were as follows:

5′ CCGAGCTCACACATTTCT 3′ (SEQ ID NO:4) and

5′ ATAAAAGGTTCTGATAAT 3′ (SEQ ID NO:5).

Using PCR, it was determined that two of the inoculated larch contained an integrated altered AHAS gene and these plants were transferred to the greenhouse for further maturation. It was also determined that six of the inoculated aspen contained an integrated altered AHAS gene and these plants were transferred to the greenhouse for further maturation.

### Example 4

Analyses of Transgenic Larch and Aspen

The altered AHAS gene copy number, integration and expression are analyzed in the transgenic larch and aspen identified in Example 3 using southern blot and northern blot analysis. Additionally, the greenhouse performance of the transgenic trees containing the altered AHAS gene is analyzed following spraying of the transgenic trees in pots with ½ field to 2× field rates, or approximately 1-3 pounds of active ingredient per acre.

1. A transgenic tree cell transformed by an altered AHAS nucleic acid, wherein expression of the nucleic acid in the tree cell results in increased resistance to an imidazolinone herbicide as compared to a wild type variety of the tree cell.

2. The transgenic tree cell of claim 1, wherein the tree is a member of a genus selected from the group consisting of a Larix genus and a Populus genus.

3. The transgenic tree cell of claim 1, wherein the tree is a Larix species selected from the group consisting of: Larix decidua, Larix lyallii, Larix kaempferi, Larix laricina, Larix occidentalis and hybrids thereof.

4. The transgenic tree cell of claim 1, wherein the tree is a Populus species selected from the group consisting of: Populus angustifolia, Populus balsamifera, Populus canadescens, Populus deltoides, Populus fremontii, Populus grandidentata, Populus tremuloides, Populus trichocarpa and hybrids thereof.

5. The transgenic tree cell of claim 1, wherein the imidazolinone herbicide is selected from the group consisting of: 2-(4-isopropyl-4-methyl-5-oxo-2-imidiazolin-2-yl)-nicotinic acid, 2-(4-isopropyl)-4-methyl-5-oxo-2-imidazolin-2-yl)-3-quinolinecarboxylic acid, 5-ethyl-2-(4-isopropyl-4-methyl-5-oxo-2-imidazolin-2-yl)-nicotinic acid, 2-(4-isopropyl-4-methyl-5-oxo-2-imidazolin-2-yl)-5-(methoxymethyl)-nicotinic acid, 2-(4-isopropyl-4-methyl-5-oxo-2-imidazolin-2-yl)-5-methylnicotinic acid, and a mixture of methyl 6-(4-isopropyl-4-methyl-5-oxo-2-imidazolin-2-yl)-m-toluate, methyl 2-(4-isopropyl-4-methyl-5-oxo-2-imidazolin-2-yl)-p-toluate and any derivative thereof.

6. The transgenic tree cell of claim 1, wherein the altered AHAS nucleic acid comprises a polynucleotide sequence selected from the group consisting of:
a) a polynucleotide as shown in SEQ ID NO:1;
b) a polynucleotide as shown in SEQ ID NO:2;
c) a polynucleotide encoding a polypeptide as shown in SEQ ID NO:3;
d) a polynucleotide comprising at least 60 consecutive nucleotides of any of a) through c); and
e) a polynucleotide complementary to the polynucleotide of any of a) through d).

7. The transgenic tree cell of claim 1, wherein the altered AHAS nucleic acid comprises a polynucleotide as shown in SEQ ID NO:2.

8. The transgenic tree cell of claim 1, wherein the altered AHAS nucleic acid comprises a polynucleotide encoding a polypeptide as shown in SEQ ID NO:3.

9. A transgenic tree comprising a tree cell according to any of claims 1-8, wherein expression of the nucleic acid in the tree results in the tree's increased resistance to an imidazolinone herbicide as compared to a wild type variety of the tree.

10. A seed produced by a transgenic tree comprising a tree cell according to any of claims 1-8, wherein the seed is true breeding for an increased resistance to an imidazolinone herbicide as compared to a wild type variety of the seed.

11. A method of producing a transgenic tree cell with an increased resistance to an imidazolinone herbicide as compared to a wild type variety of the tree cell comprising, transforming the tree cell with an expression cassette comprising an altered AHAS nucleic acid.

12. A method of producing a transgenic tree comprising,
(a) transforming a tree cell with an expression cassette comprising an altered AHAS nucleic acid, and
(b) generating a tree with an increased resistance to an imidazolinone herbicide from the tree cell.

13. The method of claim 11 or claim 12, wherein the tree is a member of a genus selected from the group consisting of a Larix genus and a Populus genus.

14. The method of claim 11 or claim 12, wherein the tree is a Larix species selected from the group consisting of: Larix decidua, Larix lyallii, Larix kaempferi, Larix laricina, Larix occidentalis and hybrids thereof.

15. The method of claim 11 or claim 12, wherein the tree is a Populus species selected from the group consisting of: Populus angustifolia, Populus balsamifera, Populus canadescens, Populus deltoides, Populus fremontii, Populus grandidentata, Populus tremuloides, Populus trichocarpa and hybrids thereof.

16. The method of claim 11 or claim 12, wherein the imidazolinone herbicide is selected from the group consisting of: 2-(4-isopropyl-4-methyl-5-oxo-2-imidiazolin-2-yl)-nicotinic acid, 2-(4-isopropyl)-4-methyl-5-oxo-2-imidazolin-2-yl)-3-quinolinecarboxylic acid, 5-ethyl-2-(4-isopropyl-4-methyl-5-oxo-2-imidazolin-2-yl)-nicotinic acid, 2-(4-isopropyl-4-methyl-5-oxo-2-imidazolin-2-yl)-5-(methoxymethyl)-nicotinic acid, 2-(4-isopropyl-4-methyl-5-oxo-2-imidazolin-2-yl)-5-methylnicotinic acid, and a mixture of methyl 6-(4-isopropyl-4-methyl-5-oxo-2-imidazolin-2-yl)-m-toluate, methyl 2-(4-isopropyl-4-methyl-5-oxo-2-imidazolin-2-yl)-p-toluate and any derivative thereof.

17. The method of claim 11 or claim 12, wherein the altered AHAS nucleic acid comprises a polynucleotide sequence selected from the group consisting of:
a) a polynucleotide as shown in SEQ ID NO:1;
b) a polynucleotide as shown in SEQ ID NO:2;
c) a polynucleotide encoding a polypeptide as shown in SEQ ID NO:3;
d) a polynucleotide comprising at least 60 consecutive nucleotides of any of a) through c); and
e) a polynucleotide complementary to the polynucleotide of any of a) through d).

18. The method of claim 11 or claim 12, wherein the altered AHAS nucleic acid comprises a polynucleotide as shown in SEQ ID NO:2.

19. The method of claim 11 or claim 12, wherein the altered AHAS nucleic acid comprises a polynucleotide encoding a polypeptide as shown in SEQ ID NO:3.

20. The method of claim 12, wherein the expression cassette further comprises a transcription initiation regulatory region and a translation initiation regulatory region that are functional in the tree.

21. A method of controlling weeds within the vicinity of a tree, comprising applying an imidazolinone herbicide to the weeds and to the tree, wherein the tree has increased resistance to the imidazolinone herbicide as compared to a wild type variety of the tree and wherein the tree comprises an altered AHAS nucleic acid.

22. The method of claim 21, wherein the tree is a member of a genus selected from the group consisting of a Larix genus and a Populus genus.

23. The method of claim 21, wherein the tree is a Larix species selected from the group consisting of: Larix decidua, Larix lyallii, Larix kaempferi, Larix laricina, Larix occidentalis and hybrids thereof.

24. The method of claim 21, wherein the tree is a Populus species selected from the group consisting of: Populus angustifolia, Populus balsamifera, Populus canadescens, Populus deltoides, Populus fremontii, Populus grandidentata, Populus tremuloides, Populus trichocarpa and hybrids thereof.

25. The method of claim 21, wherein the imidazolinone herbicide is selected from the group consisting of: 2-(4-isopropyl-4-methyl-5-oxo-2-imidiazolin-2-yl)-nicotinic acid, 2-(4-isopropyl)-4-methyl-5-oxo-2-imidazolin-2-yl)-3-quinolinecarboxylic acid, 5-ethyl-2-(4-isopropyl-4-methyl-5-oxo-2-imidazolin-2-yl)-nicotinic acid, 2-(4-isopropyl-4-methyl-5-oxo-2-imidazolin-2-yl)-5-(methoxymethyl)-nicotinic acid, 2-(4-isopropyl-4-methyl-5-oxo-2-imidazolin-2-yl)-5-methylnicotinic acid, and a mixture of methyl 6-(4-isopropyl-4-methyl-5-oxo-2-imidazolin-2-yl)-m-toluate, methyl 2-(4-isopropyl-4-methyl-5-oxo-2-imidazolin-2-yl)-p-toluate and any derivative thereof.

26. The method of claim 21, wherein the altered AHAS nucleic acid comprises a polynucleotide sequence selected from the group consisting of:
a) a polynucleotide as shown in SEQ ID NO:1;
b) a polynucleotide as shown in SEQ ID NO:2;
c) a polynucleotide encoding a polypeptide as shown in SEQ ID NO:3;
d) a polynucleotide comprising at least 60 consecutive nucleotides of any of a) through c); and
e) a polynucleotide complementary to the polynucleotide of any of a) through d).

26. The method of claim 21, wherein the altered AHAS nucleic acid comprises a polynucleotide as shown in SEQ ID NO:2.

28. The method of claim 21, wherein the altered AHAS nucleic acid comprises a polynucleotide encoding a polypeptide as shown in SEQ ID NO:3.

29. The method of claim 21, wherein the altered AHAS nucleic acid is contained within an expression cassette and the expression cassette further comprises a transcription initiation regulatory region and a translation initiation regulatory region that are functional in the tree.
